(19) United States
(12) Patent Application Publication
(10) Pub. No.: US 2022/0034836 A1
(43) Pub. Date: Feb. 3, 2022

(54) DEVICE FOR MEASURING WATER CONTENT

(71) Applicant: EPYMETRICS AG, Zürich (CH)

(72) Inventors: Peter Heeb, Balgach (CH); Monika Baur, Horn (CH); Emine Cagin, Buchs (CH); André Bernard, St. Gallen (CH)

(21) Appl. No.: 17/276,501

(22) PCT Filed: Sep. 17, 2019
(86) PCT No.: PCT/EP2019/074784

§ 371 Date: Mar. 16, 2021

(30) Foreign Application Priority Data
Sep. 17, 2018 (EP) 18194729.2

(51) Int. Cl. G01N 27/22; G01L 13/00; G01P 5/02; G01N 33/487; A61B 5/1477

(52) U.S. Cl. CPC G01N 27/223; G01L 13/00; A61B 5/14507; G01N 33/48707; A61B 5/1477; G01P 5/02

(57) ABSTRACT

Embodiments concern a high-precision, measurement device operative to measure the water content in media and/or water transport rate by media with high precision and with high dynamic range concerning the flow rate value. Based on a molecular transducer principle, captured water reacts with a reactant characterized by its ability to generate gas as a reaction product. By using an electro-chemical transducing element, an electric signal is generated in accordance with a stoichiometric volume of gas produced and water transferred, which is related to the flow rate of the circulating aqueous solution.

### CROSS REFERENCE TO RELATED APPLICATIONS

This application is a 371 application from international patent application No. PCT/EP2019/074784, which claims priority to EP patent application No. 18194729.2 filed Sep. 17, 2018, the content of which is incorporated herein by reference in its entirety.

### BACKGROUND

High-precision measurement of water content in the flow regime of less than 10 microliter/minute is crucial for many applications. Today, there are multiple state-of-the art solutions; however, each suffers from a deficiency. Many solutions require expensive, specialized equipment that are dependent on an external electrical power source and require a lab setting. Examples include, thermal mass flow meter, Doppler ultrasonic flow meters, Venturi flow meters, Coriolis flow meters just to name a few.

Thermal mass-flow meters, also called thermal anenometers, are simpler to employ. However, they are limited to a defined continuum of flow of fluid having a constant and well-known density and heat capacitance within the volume stream passing the sensor. Thermal anenometers are therefore incapable of measuring water flow in, for example, aqueous solutions of unknown composition exhibiting unknown thermo-physical parameters.

Therefore, there is a need for a high-precision measurement device that can reliably and precisely determine the flow rate of an aqueous solution, circulating in a known environment (such as a microfluidic system), that is inexpensive, and deployable in a wide variety of settings function in the above-noted flow regime.

### BRIEF DESCRIPTION OF THE DRAWINGS

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The number of elements shown in the Figures should by no means be construed as limiting and is for illustrative purposes only. The figures are listed below.

FIG. 1 is a schematic, cross-sectional side-view of a measurement device, according to some embodiments;

FIGS. 2A-2F are schematic, cross-sectional side-views of various embodiments of a transducing element employable in the measurement device of FIG. 1;

FIG. 3 is a schematic, cross-sectional side-view of a measurement device mounted on a body to measure the flow rate of water, for example, due to the discharge of fluid from matter (e.g., perspiration or sweat rate), according to some embodiments; and

FIG. 4 is a flowchart of the process steps employed by the measurement device, according to some embodiments.

### DETAILED DESCRIPTION

The following description sets forth various details to provide a thorough understanding of the invention and it should be appreciated, by those skilled in the art, that the present invention may be practiced without these specific details. Furthermore, well-known methods, procedures, and components have been omitted to highlight the present invention.

The present invention pertains to a measuring device for measuring the amount of water content in media, and/or the water transport rate caused by the flow of media, and/or the water flow rate in media, and/or water flow rate caused by the flow of media containing water.

The media may originate from a fluid source 10, which can be a static or dynamic type of source for fluid. Fluid source 10 may be reservoir, and/or a fluid stream. Fluid source 10 may for example comprise bodily and/or non-bodily sources of (e.g., flowing) media. Fluid source 10 may include matter or composition of matter such as living tissue (e.g., skin tissue), non-living tissue, synthetic material, and/or non-synthetic material.

For example, the measuring device may be operable to measure the (e.g., instantaneous) amount of water contained in media, and/or a water flow rate and/or the transport rate of water contained in flowing media (e.g., aqueous solutions).

In some examples, the measuring device may be operable to measure, respectively, the (e.g., instantaneous) amount of media based on a measured amount of water contained in the media. Further, the measuring device may be operable to measure a media flow rate, based on the measured water flow rate and/or water transport rate by the media.

Flowing media can include, for example, bodily matter or media that may contain water and that may be discharged (e.g., excreted or secreted) from and/or by an animal body. Bodily matter may include, for example, sweat, tears, saliva, urine and/or stool.

In some embodiments, the measuring device may be employed to measure the flow rate of the media containing water and/or the instantaneous amount of media containing water, based on the measured amount of water content and/or water flow rate.

The measuring device may be implemented, for example, as an electrochemical sensor that is operative to measure comparatively low or extremely-low (e.g., instantaneous) water content (e.g., the amount of water) in an (e.g., flowing) liquid volume and/or the water flow rate of water-containing liquid such as, for example, blood, sweat, saliva and/or tears. Correspondingly, the measurement device, which may herein also referred to as “sensor”, may be operable to measure sweat rate based on the amount of water contained in the sweat.

Although embodiments of the present disclosure may relate to the measurement of water flow rates of discharged bodily liquids, this should by no means be construed in a limiting manner. Accordingly, embodiments may generally pertain to performing phase-independent flow rate (e.g., mass-flow) measurement of water (or the measurement of water transport rate) contained in, for example, liquid media, gaseous media, and/or in mixed-state media such as vapor and/or solid-fluid media, by an open-system configuration, where the device can be continuously subjected to the flow of media, for example, for the instantaneous measurement of the amount water contained therein and/or for measuring the flow rate of the water contained in the (e.g., flowing or non-flowing) media.

In some embodiments, the device is configured to enable measuring the amount of water that is transported per time in flowing media, for example, independently of the state of aggregation and/or type media (mixed state or non-mixed state) and/or independently of the composition of the media. For example, the measurement device may be operably employable in conjunction with media in which the water content is comparatively high (e.g., aqueous solutions) and/or in conjunction with media in which the water content is comparatively low, for example, in which the water is only a trace component.

Optionally, the device is configured to enable measuring the amount of water that is transported per time unit in media flowing in a channel (e.g., a channel of a microfluidic device).

Optionally, the dynamic range of the same measurement device may allow for measuring the (e.g., instantaneous) amount of water contained in media and/or the flow rate of water contained in media in which the water content may be high (e.g., in aqueous solutions) as well in media in which the water content may be low (e.g., water is a trace component).

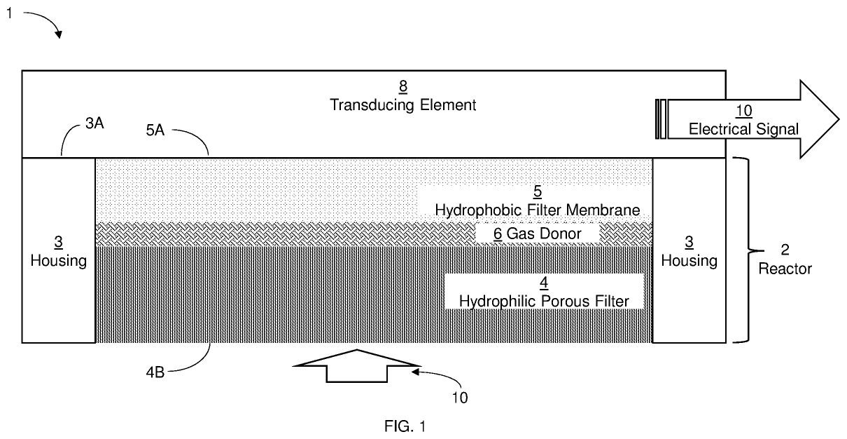

Turning now to the figures, FIG. 1 is a schematic, cross-sectional side-view of a measurement device 1 and includes, for example, two primary elements; a reactor 2 that directly or indirectly engages with and/or is in fluid communication with transducing element 8, according to an embodiment. Reactor 2 includes, for example, a (e.g., polymeric) housing 3 that may at least partially encase a hydrophilic porous filter 4 for capturing moisture and filtering impurities, a gas donor 6 characterized by its ability to generate (e.g., hydrogen) gas upon reaction with water, and a hydrophobic filter membrane 5 operative to capture unreacted water and other reactants. Transducing element 8 is operative to transduce the gas into an electrical signal 10 and can be implemented in various configurations as will be further discussed.

In some embodiments, hydrophilic porous filter 4 can be disposed such to be in fluid communication with a fluid source 10. Accordingly, measurement device 1 may be operable to measure an amount of media by measuring the amount of water contained in the media, and/or the flow rate of media by measuring the water transport rate by the flowing media in an open-system setup, for example, for the (e.g., continuous) flow rate measurement of bodily fluids being discharged.

In some embodiments, gas donor 6 may be disposed between hydrophilic porous filter 4 and hydrophobic filter membrane 5. In some embodiments, hydrophobic filter membrane 5 may be disposed between gas donor 6 and transducing element 8. Optionally, hydrophilic porous filter 4, hydrophobic filter membrane 5, gas donor 6 and/or transducing element 8 may be disposed in a layered manner. Optionally, housing 3 may extend laterally from an upper surface 5A of hydrophobic filter membrane 5 to a lower surface 4B of hydrophilic porous filter 4. Housing 3 may therefore laterally encase hydrophilic porous membrane 4, gas donor 6 and hydrophobic filter membrane 5. In some embodiments, as exemplified in FIG. 1, transducing element 8 may extend over the upper surface 5A of hydrophobic filter membrane 5 and the upper surface or edge 3A of housing 3. Optionally, the upper surfaces 3A and 5A may be flush. In some other embodiments, the upper surfaces 3A and 5A may not be flush. Further, in some other embodiments, transducing element 8 may be sized such to not extend over the later edges of hydrophobic filter membrane 5. Optionally, housing 3 may be arranged to laterally extend over the lateral edges of transducing element 8.

In some embodiments, the lower surface 4B of hydrophilic porous filter 4 may be exposed to the environment, for example, to allow for the continuous measurement of water content and/or water flow rate in liquid that is, for example, being discharged by an animal body, e.g., from the skin of a living mammalian

In some embodiments, measurement device 1 may comprise a fastener for allowing operably (and optionally, removably) coupling measurement device 1, for example, with an animal body such to allow for the measurement of water content and/or water flow rate contained in bodily fluid discharged from and/or by animal body. The fastener may include, for example, an adhesive, staple, tack, suture, and/or the like.

In some embodiments, measurement device 1 may be configured as and/or incorporated in a patch-like structure.

In some embodiments, measurement device 1 may be an implantable measurement device 1.

In some embodiments, measurement device 1 may be operably engaged with a skin surface portion of an animal body. In some embodiments measurement device 1 may be operably coupled with a tissue portion of a body organ in addition to the skin such as, for example, the inner surface and/or outer surface of the gastrointestinal tract, the urinary tract; with the peritoneum, and/or the like.

In some embodiments, measurement device 1 may be operably coupled with a catheter and/or any other medical device for measuring water content being present and/or flowing therein.

Hydrophilic porous filter 4 provides a constant flow resistance to both liquid and gas states in the above-noted flow regime, and the hydrophilic properties do not hinder passage or facilitate passage of liquid water at the low pressures that may be associated with low flow rate regimes towards gas donor 6. For example, the hydrophilic properties of porous filter 4 may facilitate the passage of water-containing liquid from fluid source 10 towards gas donor 6. Filter 4 may be made, for example, of glass, ceramic, metal, and/or cellulose and may, for example, have a pore size of 450 nm enabling passage of liquid and vapor while filtering particles or salt compounds.

In another embodiment, Hydrophilic porous filter 4 is implemented as Anodic Aluminum Oxide or Anodic Titanium Oxide with a pore size of, e.g., 5-500 nm.

As shown, gas donor 6 is disposed at the downstream side of filter 4 to enable reaction with filtered water vapor conveyed by pressure exerted by the water source through primary porous filter 4. In certain filters 4, the liquid water is also conveyed through via capillary action from the outside to the inside of upstream reactor 2.

In some embodiments, CAH₂ is employed as the gas donor. Alternative donors of hydrogen include, for example, metal-hydrides such as MgH₂, NaAlH₄, LiAlH₄, LiH, LiBH₂, LiBH₄, non-metal hydrides, and/or some carbohydrates. The gas donor can act as a battery substitute and may define the upper limit of the cumulative electrical power that can be generated.

Hydrophobic filter membrane 5 is implemented as a barrier to enclose the CaH₂, H₂O and Ca(OH)₂ and prevent them from entering the downstream fuel cell, in a certain embodiment. In another embodiment, filter membrane 5 is implemented as combination cellulose/polyester cloth.

Transducing element 8 translates the gas into an electrical signal by any of a variety of transducing element embodiments, as will be now discussed.

The measurement device 1 may be configured such that hydrogen gas generated thereby is released into the environment. In this way, hydrogen gas can be continuously generated in response to (e.g., continuously) subjecting hydrophyilic porous filter 4 with (e.g., flowing) media that may contain water.

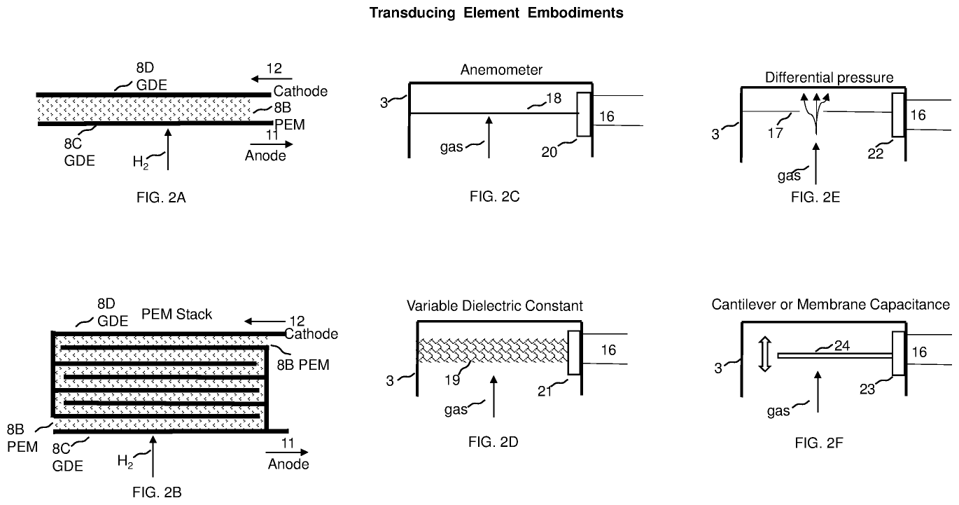

FIGS. 2A-2F are schematic, cross-sectional side-views of various embodiments of transducing element 8.

Specifically, FIG. 2A depicts an embodiment of transducing element 8 implemented as a proton-exchange membrane (PEM) fuel cell in which hydrogen contacting a downstream gas diffusion electrode (GDE) 8C is oxidized and the electrons exit the cell on the anode side 11 through an electrical conductor. The resulting cations traverse PEM 8B. At GDE 8D the hydrogen ions recombine with electrons and form water through reaction with oxygen. In a certain embodiment PEM 8B is implemented as Nafion.

FIG. 2B depicts an embodiment of transducing element 8 employing a polymer stack of the PEM fuel cells.

FIG. 2C depicts an embodiment of transducing element 8 that employs a heating filament 18 cooled by the flow of gas. As shown, at least some of the gas is directed over a resistance heated wire 18. The resulting change in resistance or temperature distribution profile is measured by circuitry 20 and output, e.g., via leads 16 and/or a wireless transmitter. It should be appreciated that the above noted deficiencies of an anemometer are removed through conversion of any fluid media (e.g., aqueous solution) operably engaging with the device into a pure gas. It is noted that fluid media may be characterized by its composition-dependent density and heat capacity.

FIG. 2D depicts an embodiment of transducing element 8 that employs a porous material 19 whose dielectric constant changes as it fills with gas. The resulting change in capacitance is processed by circuitry 21 and outputs a signal via leads 16 and/or a wireless transmitter. Zeolite is an example of a such a porous material.

FIG. 2E depicts an embodiment of transducing element 8 that employs a differential pressure sensor to transduce gas pressure into an electrical signal. As shown, a differential pressure is created as gas passes through orifice 17 and is transduced into an electric signal by circuitry 22, and signal output, e.g., via leads 16 and/or a wireless transmitter.

FIG. 2F depicts an embodiment of transducing element 8 that employs a cantilever or stretchable membrane configured to deflect responsively to the local pressure field generated by the gas-stream. As shown, gas applies a pressure to flexible element 24 and the resulting deformation is quantified through an electro-mechanical transducer or circuitry 23, and a signal output, e.g., via leads 16 and/or a wireless transmitter.

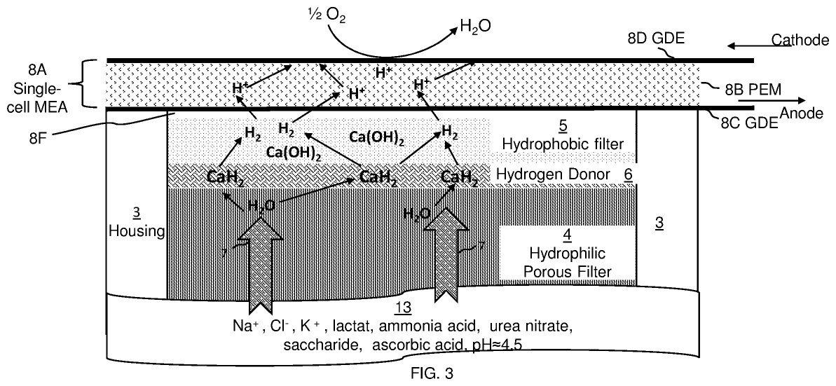

FIG. 3 depicts an embodiment of measurement device 1 applied as a sweat gauge mounted to sweating skin 13.

Water and other sweat constituents are captured by primary hydrophilic porous filter 4, the non-aquatic constituents are filtered out, and the remaining water content conveyed downstream where it contacts the hydrogen donor CaH₂ 6 disposed at the downstream edge of primary filter 4. There the CaH₂ reacts with water to form a stoichiometric volume of hydrogen that creates a pressure gradient driving the hydrogen through secondary filter 5. Water filter 5 filters unreacted water, CaH₂ and Ca(OH)₂ and has a low flow resistance relative to primary filter 4. This filtering may become increasingly significant in protecting transducing element 8 as reaction efficiency diminishes with time and the quantity of unreacted water increases. The hydrogen continues downstream and contacts transducing element 8 implemented in this example as a single-cell membrane electrode assembly (MEA) having a proton exchange membrane PEM 8B sandwiched between two gas diffusion electrodes GDEs 8C and 8D, as noted above. Each of GDEs 8C and 8D has an electrically conductive supporting cloth enabling gas distribution and an electrode with a catalyst where the chemical reactions occur. The catalyst coated surfaces are in contact with PEM electrolyte 8B.

As shown, GDE 8C hydrogen is oxidized to cations H+ and the electrons leave measurement device 1 at anode 11. The cations pass the solid-state electrolyte PEM 8B and the oxygen is reduced and combines with cations to produce water at cathode 12, as noted above.

The PEM 8B is a gas selective permeable membrane resulting in a hydrogen and oxygen gradient across the membrane thickness. It acts as convey path for protons supply the GDE 8D with protons H+, while blocking oxygen and ions thereof. The GDE 8D in contact with the PEM promotes a high conversion rate of the protons to water.

MEA 8A is in communication with gas distribution channel 8F to maximize hydrogen contact with to the membrane surface. A high conversion rate is obtained by using MEAs. Non-converted excess hydrogen can leave the system after passing membrane surface 8D in a certain embodiment.

In another embodiment, a bypass channel (not shown) directs a known, fixed fraction of hydrogen directly out of housing 3 and does not produce an electrical signal to prevent saturation of the fuel cell and facilitate miniaturization of MEA 8A. Measurement device 1 can be self-actuated and deactivated in accordance with stoichiometric limitation set by the amount of water available.

As taught, the required precision measurement is achieved through the capture of sweat in static and/or any flowing state and the conversion of, for example, sweat, based on the amount of water contained therein, into a corresponding collective electrical signal.

Other applications include, inter alia, measurement of (e.g., minute) water content and/or flow rates in media that includes flowing, partially flowing or non-flowing solid or semi-solid materials and/or composition of matter, including, for example, soil, concrete, apparel, polymeric materials, non-polymeric materials, organic matter, and/or the like. Additional examples of media for which the water content may be measured includes the atmosphere. Further applications can include determining the (e.g., water-) permeability of objects, their time dependent evaporation behavior, and/or additional characteristics.

In yet other applications, precision measurement of water content (including, for example, the flow rate of circulating media) is achievable independently of the Reynold's numbers characteristic of laminar and turbulent flow regimes.

Furthermore, in addition to its measuring capacity, the sensor also generates harnessable electricity.

Measurement device 1 may be comparatively efficient and effective for static and non-static states and have a high sensitivity down to flow rates of the less than, for example, 10 uL/min. Measurement device 1 is also effective for liquid, gaseous, and vapor states over a temperature range like, for example, 5-40° C., has a fast response time of some seconds, has a slew rate of, e.g., 3-5 seconds to reach the nominal output power, and has a comparatively low cost. Its dynamic range spans, e.g., at least five orders of magnitude and is highly selective in that its capable of identifying and measuring the amount of water included in any media that may comprise, for example, aqueous solutions among a variety of other compositions.

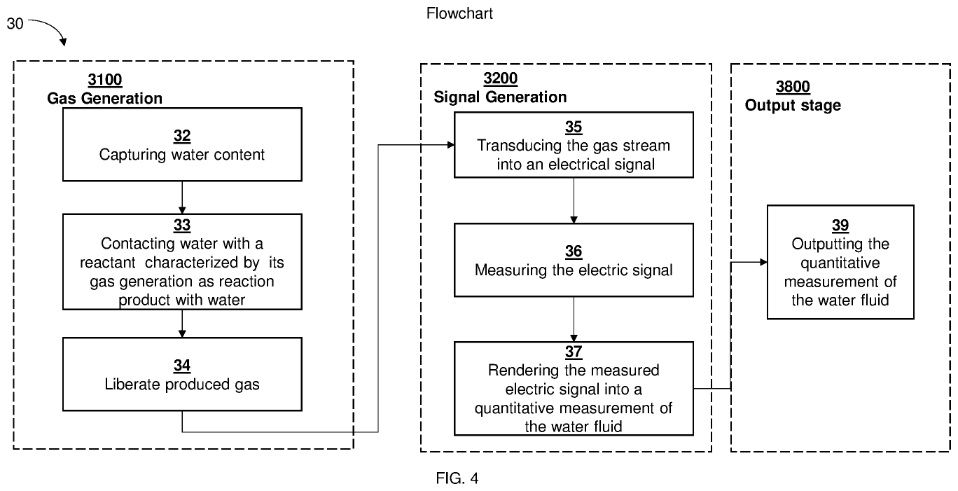

FIG. 4 is a flowchart of the processing steps employed by the measurement device (e.g., water sensor) and can be divided into three stages, gas generation 3100, signal generation 3200, and output 3800.

Specifically, in gas generation stage 3100, water is captured at step 32 as a liquid, gas or vapor with a hydrophilic material, as noted above. In step 33, the water is contacted with a reactant characterized by its gas generation properties as a reaction product. In step 34 the liberated gas is conveyed through a hydrophobic membrane 5 while filtering unreacted water and reacted CaH₂.

In stage 3200, the liberated gas is transduced into an electrical signal at step 35 through any of the transducing element embodiments noted above. In step 36, the signal is measured either as a current or a voltage in accordance with the type of transducing element employed. In step 37, the measured signal is rendered into a quantitative measurement of the flow rate of water fluid in accordance with a given reaction conversion.

In the single-step output stage 3800, the quantitative measurement is output at step 39, for example, to a display screen.

### Additional Examples

Some examples concern a high-precision, measurement device operative to measure the water content in media and/or water transport rate by media with high precision and with high dynamic range concerning the flow rate value. Further examples concern a measurement device that is operative to measure an (e.g., instantaneous) amount of media and/or media flow rate, based on water content in the media and/or a water transport rate by the media.

Based on a molecular transducer principle, captured water reacts with a reactant characterized by its ability to generate gas as a reaction product. By using an electro-chemical transducing element, an electric signal is generated in accordance with a stoichiometric volume of gas produced and water transferred, which is related to the flow rate of the circulating aqueous solution.

Example 1 is a method for quantifying the content of water (e.g., flow rate of any flowing media (e.g., aqueous solution), the method comprising: contacting water fluid with a reactant hydrogen donor; capturing a liberated hydrogen gas stream having a stoichiometric equivalent of the water fluid; transducing the hydrogen gas stream into an electrical signal; determining a characteristic of the water fluid in accordance with the electric signal; and providing an output descriptive of the determined water fluid characteristic.

Example 2 includes the subject matter of example 1 and, optionally, wherein the media is a pure liquid.

Example 3 includes the subject matter of example 1 or example 2 and, optionally, wherein the media is gas.

Example 4 includes the subject matter of any one of the examples 1 to 3 and, optionally, wherein the media is in a mixed state comprising, for example, vapor and/or a solid-liquid media.

Example 5 includes the subject matter of any one of the examples 1 to 4 and, optionally, wherein the reactant is implemented as metallic or non-metallic hydride.

Example 6 includes the subject matter of example 5 and, optionally, wherein the metallic hydride is selected from the group consisting of MgH₂, NaAlH₄, LiAlH₄, LiH, LiBH², and LiBH₄.

Example 7 includes the subject matter of example 5 or 6 and, optionally, wherein the non-metallic hydride is implemented as CaH₂.

Example 8 includes the subject matter of any one of the examples 1 to 7 and, optionally, wherein the transducing the hydrogen stream into an electrical signal is implemented by driving a fuel cell with the hydrogen stream to yield a corresponding change in current flow.

Example 9 includes the subject matter of any one of the examples 1 to 8 and, optionally, wherein the transducing the hydrogen stream into an electrical signal is implemented by permeating a porous dielectric with the hydrogen stream so as to yield a corresponding change in electrical capacitance of the porous dielectric. Optionally, one can use the change of the thermal conductivity by using a low conductive porous material; (e.g. aerogel, porous silica).

Example 10 includes the subject matter of example 9 and, optionally, wherein the porous dielectric is implemented as a zeolite.

Example 11 includes a measurement device (1) for quantifying water content in flowing or non-flowing media, the measurement device (1) comprising: a reactor (2) including a reactant hydrogen donor, wherein the reactor (2) is configured to liberate a hydrogen stream having a stoichiometric equivalent to water; the reactant hydrogen donor having an ability to liberate hydrogen gas upon reaction with water. The measurement device (1) may further include, optionally, a transducing element configured to transduce the hydrogen stream into an electrical signal; circuitry configured to determine a quantity of the water fluid in accordance with the electrical signal. Optionally, the measurement device (1) may comprise an output device configured to output the quantity of the water fluid. Such output may comprise a visual, audible, haptic, or digital output, or a combination of them in accordance with the particular embodiment.

Example 12 includes the subject matter of example 11 and, optionally, wherein the media is implemented as liquid, gas, vapor and/or a liquid-solid media.

Example 13 includes the subject matter of examples 11 or 12 and, optionally, wherein the reactant hydrogen donor is implemented as metallic or non-metallic hydride.

Example 14 includes the subject matter of any one of the examples 11 to 13 and, optionally, wherein the metallic hydride is selected from the group consisting of MgH2, NaAlH4, LiAlH4, LiH, LiBH2, and LiBH4.

Example 15 includes the subject matter of example 13 and, optionally, wherein the metallic hydride is implemented as CaH₂.

Example 16 includes the subject matter of any one of the examples 11 to 15 and, optionally, wherein the transducing element is implemented as a fuel cell driven by the hydrogen stream.

Example 17 includes the subject matter of example 16 and, optionally, wherein the fuel cell includes a proton-exchange membrane (PEM).

Example 18 includes the subject matter of any one of the examples 11 to 17 and, optionally, wherein the transducing element is implemented as porous dielectric whose capacitance changes in accordance with a degree of permeation of the hydrogen stream.

Example 19 includes the subject matter of example 18 and, optionally, wherein the porous dielectric is implemented as a zeolite.

Example 20 includes the subject matter of any one of the examples 11 to 19 and, optionally, wherein the transducing element is implemented as a pressure sensor configured to generate the electric signal responsively to a differential pressure generated by the hydrogen stream.

Example 21 includes the subject matter of any one of the examples 11 to 20, and optionally, wherein the transducing element is implemented as an anemometer configured to generate the electric signal in accordance with gas flow of the hydrogen stream.

Example 23 concerns a measuring device (1) for measuring water content in a media, comprising: a reactor (2) comprising a reactant gas donor (6), wherein the reactor (2) is configured to liberate a hydrogen stream having a stoichiometric equivalent to water in the media, the reactant gas donor (6) having an ability to liberate hydrogen gas upon reaction with water; and a transducing element (8) configured to transduce the hydrogen stream into an electrical signal, wherein the reactor (2) is configured such that the reactant gas donor (6) can be continuously subjected to a flow of water-containing media.

Example 24 includes the subject matter of example 23 and, optionally, further comprising circuitry (20, 21, 22, 23) that is configured to determine a value related to a characteristic of water fluid in accordance with the electrical signal; and an output device configured to output the quantity of the water fluid.

Example 25 includes the subject matter of examples 23 or 24 and, optionally, wherein the water fluid is implemented as liquid, gas, and/or vapor.

Example 26 includes the subject matter of any one or more of the examples 23 to 25 and, optionally, wherein the reactant gas donor (6) is implemented as metallic or non-metallic hydride.

Example 27 includes the subject matter of example 26 and, optionally, wherein the metallic hydride is selected from the group consisting of MgH2, NaAlH4, LiAlH4, LiH, LiBH2, and LiBH4.

Example 28 includes the subject matter of example 27 and, optionally, wherein the metallic hydride is implemented as CaH2.

Example 29 includes the subject matter of any one or more of examples 23 28 and, optionally, wherein the transducing element (8) is implemented as a fuel cell (8A, 8B) driven by the hydrogen stream.

Example 30 includes the subject matter of example 29 and, optionally, wherein the fuel cell (8A, 8B) includes a proton-exchange membrane (PEM).

Example 31 includes the subject matter of any one of examples 23 to 30 and, optionally, wherein the transducing element (8) is implemented as porous dielectric (19) whose capacitance changes in accordance with a degree of permeation of the hydrogen stream.

Example 32 includes the subject matter of example 31 and, optionally, wherein the porous dielectric (19) is implemented as a zeolite.

Example 33 includes the subject matter of any one or more of examples 23 to 32 and, optionally, wherein the transducing element (8) is implemented as a pressure sensor configured to generate the electric signal responsively to a differential pressure generated by the hydrogen stream.

Example 34 includes the subject matter of any one or more of examples 23 to 33 and, optionally, wherein the transducing element (8) is implemented as an anemometer configured to generate the electric signal in accordance with gas flow of the hydrogen stream.

Example 35 includes the subject matter of any one or more of examples 23 to 34 and, optionally, configured such that the gas donor (6) makes direct or indirect contact with an animal body for measuring the water content contained in bodily media discharged by the animal body and, wherein, the bodily media comprises, for example, sweat, blood, saliva, tears, urine and/or stool.

Example 36 includes the subject matter of any one or more of the examples 23 to 35 and, optionally, further comprising a hydrophilic porous filter (4), wherein the gas donor (6) is disposed within and/or layered above the hydrophilic porous (4), wherein the hydrophilic porous filter (4) can be in fluid communication with a fluid (10).

Example 22 concerns the use of a measurement device according to any one of the examples 11 to 21 or 23 to 36.

Example 37 includes a method for quantifying a characteristic related to water, the method comprising:

contacting water fluid with a reactant gas donor for generating hydrogen gas; capturing a liberated hydrogen gas stream having a stoichiometric equivalent of the water fluid; transducing the hydrogen gas stream into an electrical signal; determining an amount of water fluid in accordance with the electric signal; and releasing the captured hydrogen gas stream for allowing repeating the step of contacting water fluid with the reactant gas donor for generating hydrogen gas.

Example 38 includes the subject matter of Example 37 and, optionally, wherein the water fluid is implemented as a liquid, vapor, solid-fluid media and/or a gas.

Example 39 includes the use of any one of the measuring devices (1) of examples 23 to 36.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise specified, the terms ‘about’ and/or ‘close’ with respect to a magnitude or a numerical value may imply to be within an inclusive range of −10% to +10% of the respective magnitude or value.

“In fluid communication with” means “indirectly or directly in fluid communication with”.

“Coupled with” means “indirectly or directly coupled with”.

It should be appreciated that combination of features disclosed in different embodiments, examples, and/or the like, are also included within the scope of the present invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A measuring device for measuring water content in a media, comprising:
a reactor comprising a reactant gas donor, wherein the reactor is configured to liberate a hydrogen stream having a stoichiometric equivalent to water in the media, the reactant gas donor having an ability to liberate hydrogen gas upon reaction with water; and
a transducing element configured to transduce the hydrogen stream into an electrical signal,
wherein the reactor is configured such that the reactant gas donor (6) can be continuously subjected to flow of water-containing media.

2. The measuring device of claim 1, further comprising circuitry that is configured to determine a value related to a characteristic of water fluid in accordance with the electrical signal; and an output device configured to output the quantity of the water fluid.

3. The measuring device of claim 1, wherein the water fluid is implemented as liquid, gas, and/or vapor.

4. The measuring device of claim 1, wherein the reactant gas donor is implemented as metallic or non-metallic hydride.

5. The measuring device of claim 4, wherein the metallic hydride is selected from the group consisting of MgH2, NaAlH4, LiAlH4, LiH, LiBH2, and LiBH4.

6. The measuring device of claim 5, wherein the metallic hydride is implemented as CaH2.

7. The measuring device of claim 1, wherein the transducing element is implemented as a fuel cell driven by the hydrogen stream.

8. The measuring device of claim 7, wherein the fuel cell includes a proton-exchange membrane (PEM).

9. The measuring device of claim 1, wherein the transducing element is implemented as porous dielectric whose capacitance changes in accordance with a degree of permeation of the hydrogen stream.

10. The measuring device of claim 9, wherein the porous dielectric is implemented as a zeolite.

11. The measuring device of claim 1, wherein the transducing element is implemented as a pressure sensor configured to generate the electric signal responsively to a differential pressure generated by the hydrogen stream.

12. The measuring device of claim 1, wherein the transducing element is implemented as an anemometer configured to generate the electric signal in accordance with gas flow of the hydrogen stream.

13. The measuring device of claim 1, configured such that the gas donor makes direct or indirect contact with an animal body for measuring the water content contained in bodily media discharged by the animal body and, wherein, the bodily media comprises, for example, sweat, blood, saliva, tears, urine and/or stool.

14. The measuring device of claim 1, further comprising a hydrophilic porous filter, wherein the gas donor is disposed within and/or layered above the hydrophilic porous filter, wherein the hydrophilic porous filter can be in fluid communication with the fluid source.

15. A method for quantifying a characteristic related to water, the method comprising:
contacting water fluid with a reactant gas donor for generating hydrogen gas;
capturing a liberated hydrogen gas stream having a stoichiometric equivalent of the water fluid;
transducing the hydrogen gas stream into an electrical signal;
determining an amount of water fluid in accordance with the electric signal; and
releasing the captured hydrogen gas stream for allowing repeating the step of contacting water fluid with the reactant gas donor for generating hydrogen gas.

16. The method of claim 15, wherein the water fluid is implemented as a liquid, vapor and/or a gas.

17. (canceled)
